(19) United States
(12) Patent Application Publication
(10) Pub. No.: US 2024/0415449 A1
(43) Pub. Date: Dec. 19, 2024

(54) DEVICE AND METHOD FOR MONITORING SLEEP STATE OF COMMUNITY, AND RECORDING MEDIUM FOR PERFORMING METHOD

(71) Applicant: KOREA INSTITUTE OF SCIENCE AND TECHNOLOGY, Seoul (KR)

(72) Inventors: Jee Hyun CHOI, Seoul (KR); Bowon KIM, Seoul (KR); Jung Young KIM, Daejeon (KR)

(73) Assignee: KOREA INSTITUTE OF SCIENCE AND TECHNOLOGY, Seoul (KR)

(21) Appl. No.: 18/585,134

(22) Filed: Feb. 23, 2024

(30) Foreign Application Priority Data
Jun. 14, 2023 (KR) 10-2023-0076322

(51) Int. Cl. A61B 5/00; A61B 5/369; A61B 5/389

(52) U.S. Cl. CPC A61B 5/4809; A61B 5/0006; A61B 5/369; A61B 5/389; A61B 5/4812; A61B 2503/40

(57) ABSTRACT

A device for monitoring a sleep state of a community mounted on each measurement target forming the community includes an electrode unit configured to measure biosignals including an electroencephalogram (EEG) and electromyography (EMG) of each measurement target forming the community, a control unit configured to compare the measured EEG and

EMG with critical values and classify sleep and wake states of the measurement target, an indicating unit configured to emit light in different colors according to the sleep and wake states of the measurement target, and a recording and communication unit configured to store or transmit the measured EEG and EMG to an external device. Therefore, by measuring a sleeping behavior of each entity of the community and analyzing and visualizing the sleep and wake states in real time, it is possible to efficiently observe the interaction between sleep and an environment.

### DESCRIPTION OF GOVERNMENT-SPONSORED RESEARCH

This study was conducted with the support of the basic personal research (the Ministry of Science and ICT) of the Ministry of Science and ICT [source technology development for neuroscience of the collective brains, project identification number: 1711157654, detailed project number: 2022R1A2C3003901].

### CROSS REFERENCE TO RELATED APPLICATION

The present application claims the benefit under 35 USC 119(a) of Korean Patent Application No. 10-2023-0076322 filed Jun. 14, 2023, in the Korean Intellectual Property Office, the entire disclosure of which is incorporated herein by reference for all purposes.

### BACKGROUND OF THE INVENTION

### Field of the Invention

The present disclosure relates to a device and method for monitoring a sleep state of a community, and a recording medium for performing the method, and more specifically, to a technology of measuring a biosignal of each entity of a community (e.g., a group of mouse living in a natural group) and observing the interaction between sleep and an environment by determining sleep and wake states in real time.

### Description of the Related Art

A long-standing problem in brain and social science is to understand the neural mechanism that underlie a complex social group behavior.

Although these studies require neural processing, integrated solutions for connecting brain activity to specific social behaviors are still largely insufficient.

Conventional studies have described that an individual behavior in a group is an individual behavior in a group that appears in a frame such as collective movement (e.g. migration, defensive aggregation, or synchronized reproduction), a local behavior (e.g. competition, mating, or parenting), and a population structure (e.g., hierarchy) in a general evolutionary context.

A collective behavior is not a simple sum of the behaviors of entities, but research on the neural mechanism of a social behavior requires comparative measurement and analysis at an individual brain level.

Although these approaches emphasize causation between neural elements and a social behavior, it is very difficult to infer the collective behavior, which mostly results from the communication and interaction of entities, from knowledge of components thereof.

Moreover, many global attributes of social groups are a result of coordinated interactions between group members, and statistical results from single-brain studies are not satisfactory for characterizing social interactions.

Meanwhile, brain regions and neural circuits together with behavioral levels are reported to depend on the social context in which two or more entities interact. This is because most behaviors are results of intuitive interactions between group members.

Therefore, direct observation of brains and behaviors is required to more satisfactorily describe the neurodynamic basis of a complex social behavior, and it is necessary to observe the brain activity of multiple entities simultaneously to study social behaviors. In other words, the brain activity of each entity should be independently specified and observed simultaneously in a group.

In particular, sleep is known as be widely influenced by genes, waking life patterns including social interactions, environments, and the like. In addition, in natural situations, mouse shows various interactions and tend to sleep in a group when sleeping.

However, current mouse sleep experiments are mainly limited to arranging one entity in a small space and connecting a measuring line to a head to observe sleep and wake states. In this case, it is difficult for the mouse to move freely, and there is a limit in that only single-entity test is possible. In addition, since the sleep and wake states should be reviewed after measurement, there is a problem in that it is difficult to determine the sleep state based on only images, and the accuracy is reduced as the number of entities increases.

### Documents of Related Art

(Patent Document 1) KR No. 10-2011126 B1

### SUMMARY OF THE INVENTION

Therefore, the present disclosure has been devised in consideration of this point and is directed to providing a device for monitoring a sleep state of a community.

The present disclosure is also directed to providing a method of monitoring a sleep state of a community.

The present disclosure is also directed to providing a recording medium in which a computer program for performing the method of monitoring the sleep state of the community is recorded.

In order to achieve an object of the present disclosure, a device for monitoring a sleep state of a community mounted on each measurement target forming the community according to one embodiment includes an electrode unit configured to measure biosignals including an electroencephalogram (EEG) and electromyography (EMG) of each measurement target forming the community, a control unit configured to compare the measured EEG and EMG with critical values and classify sleep and wake states of the measurement target, an indicating unit configured to emit light in different colors according to the sleep and wake states of the measurement target, and a recording and communication unit configured to store or transmit the measured EEG and EMG to an external device.

In the embodiment of the present disclosure, the control unit may determine that when a value of the measured EMG of the measurement target is larger than a first critical value, the measurement target is in a wake state, determine that when the measured EMG value of the measurement target is smaller than a second critical value and a parietal EEG value is larger than a third critical value, the measurement target is in a REM sleep state, determine that when the measured EMG value of the measurement target is smaller than the second critical value and a frontal EEG value is smaller than a fourth critical value, the measurement target is in a NREM sleep state, and determine that when the measured EMG value of the measurement target is smaller than the first critical value and the frontal EEG value is larger than the fourth critical value, the measurement target is in the NREM sleep state.

In the embodiment of the present disclosure, the control unit may determine that when the measured EMG value of the measurement target is larger than or equal to the first critical value and the frontal EEG value is smaller than or equal to the fourth critical value, the measurement target is in the wake state or unknown state.

In the embodiment of the present disclosure, the first critical value may have a larger value than the second critical value.

In the embodiment of the present disclosure, the control unit may use programmed and pre-stored critical values.

In the embodiment of the present disclosure, the control unit may use the critical values by setting the critical values based on biosignals measured for a predetermined time.

In the embodiment of the present disclosure, the control unit may update each critical value at a predetermined period and use a fixed critical value when the critical value is not out of a preset range.

In the embodiment of the present disclosure, the update of the critical value may be indicated by using an indicating unit while the critical values are being updated.

In the embodiment of the present disclosure, the indicating unit may include a first light-emitting unit configured to indicate a position of the measurement target, a second light-emitting unit configured to indicate the wake state, a third light-emitting unit configured to indicate the REM sleep state, and a fourth light-emitting unit configured to indicate the NREM sleep state.

In the embodiment of the present disclosure, the electrode unit may further collect at least one biosignal among respiration, body temperature, heart rate, blood pressure, respiration, blood sugar, and electrocardiogram of each measurement target.

In order to achieve another object of the present disclosure, a method of monitoring a sleep state of a community according to one embodiment includes measuring biosignals including an electroencephalogram (EEG) and electromyography (EMG) of a measurement target using a device for monitoring the sleep state of the community mounted on each measurement target forming the community, comparing the measured EEG and EMG with critical values and classifying sleep and wake states of the measurement target, emitting light in different colors according to the sleep and wake states of the measurement target, and analyzing sleep patterns of the community based on images of the community and a change in light-emitting color.

In the embodiment of the present disclosure, the classifying of the sleep and wake states of the measurement target may include determining that when a value of the measured EMG of the measurement target is larger than a first critical value, the measurement target is in a wake state, determining that when the measured EMG value of the measurement target is smaller than a second critical value and a parietal EEG value is larger than a third critical value, the measurement target is in a REM sleep state, determining that when the measured EMG value of the measurement target is smaller than the second critical value and a frontal EEG value is smaller than the fourth critical value, it is determined that the measurement target is in a NREM sleep state, and determining that when the measured EMG value of the measurement target is smaller than the first critical value and the frontal EEG value is larger than the fourth critical value, the measurement target is in the NREM sleep state.

In the embodiment of the present disclosure, the classifying of the sleep and wake states of the measurement target may further include determining that when the measured EMG value of the measurement target is larger than or equal to the first critical value and the frontal EEG value is smaller than or equal to the fourth critical value, the measurement target is in the wake state or unknown state.

In the embodiment of the present disclosure, in the classifying of the sleep and wake states of the measurement target, programmed and pre-stored critical values may be used.

In the embodiment of the present disclosure, the classifying of the sleep and wake states of the measurement target may include setting critical values based on biosignals measured for a predetermined time, updating each critical value at a predetermined period, and fixing the critical value when each critical value is not out of a preset range.

In the embodiment of the present disclosure, the emitting of the light in different colors according to the sleep and wake states of the measurement target may include emitting light in a first light-emitting color for indicating a position of the measurement target, emitting light in a second light-emitting color for indicating the wake state, emitting light in a third light-emitting color for indicating the REM sleep state, and emitting light in a fourth light-emitting color for indicating the NREM sleep state.

In the embodiment of the present disclosure, the emitting of the light in different colors according to the sleep and wake states of the measurement target may further include emitting light in a fifth light-emitting color during the update of the critical value.

In the embodiment of the present disclosure, the method of monitoring the sleep state of the community may further include storing or transmitting the measured EEG and EMG to an external device.

In the embodiment of the present disclosure, in the measuring of the biosignals, at least one biosignal among respiration, body temperature, heart rate, blood pressure, respiration, blood sugar, and electrocardiogram of each measurement target may be further collected.

In order to achieve still another object of the present disclosure, a computer program for performing the method of monitoring the sleep state of the community is recorded on a computer-readable non-transitory storage medium according to one embodiment.

According to the method of monitoring the sleep state of the community, it is possible to measure the sleeping behavior of each entity of the community and observe the interaction between sleep and the environment in a short time by analyzing the sleep and wake states in real time. In addition, by visualizing the sleep and wake states analyzed in real time using light emitting diode (LED) colors or the like, it is possible to immediately observe the sleep and wake states and the positions and behaviors of the entities together through images.

Therefore, in the case of testing the ecology of the sleep activities of the community, when post-analysis rather than real-time analysis is applied to simultaneously observe the sleep and wake states and the positions and behaviors of the entities as the area of the space and the number of entities increase, it is possible to resolve the disadvantage that the number of entities that may be measured is limited by an increase in the workload for matching the video tracking of the individual entity with the measured brain waves increases.

### BRIEF DESCRIPTION OF THE DRAWINGS

FIG. 1 is a schematic diagram of a system for monitoring a sleep state of a community according to the present disclosure.

FIG. 2 is a block diagram of a device for monitoring a sleep state of a community according to one embodiment of the present disclosure.

FIG. 3 is a view illustrating an example in which an electrode unit of FIG. 2 is positioned.

FIG. 4 is a view for describing data collected by the electrode unit of FIG. 2.

FIG. 5 is a graph for describing that a control unit of FIG. 2 sets a critical value.

FIG. 6 is a view for describing a process of setting the critical value by the control unit of FIG. 2.

FIG. 7 is a view illustrating an example of a display unit of FIG. 2.

FIG. 8 is a flowchart of a method of monitoring a sleep state of a community according to one embodiment of the present disclosure.

FIG. 9 is a detailed flowchart of a process of classifying sleep and wake states of a measurement target using critical values in FIG. 8.

### DETAILED DESCRIPTION OF THE INVENTION

Detailed descriptions of the present disclosure to be described below will be made with reference to the accompanying drawings, which illustrate specific embodiments in which the present disclosure may be carried out as examples. These embodiments are described in detail so that those skilled in the art may sufficiently carry out the present disclosure. It should be understood that various embodiments of the present disclosure differ from one another but are not necessarily mutually exclusive. For example, specific shapes, structures and characteristics described herein can be implemented in other embodiments without departing from the spirit and scope of the present disclosure in connection with one embodiment. In addition, it should be understood that positions or arrangement of individual components in each disclosed embodiment may be changed without departing from the spirit and scope of the present disclosure. Therefore, the detailed descriptions to be described below are not intended to be taken in a limiting sense, and the scope of the present disclosure is limited only by the appended claims together with all equivalents of the claims when appropriately described. Similar reference numbers in the drawings refer to identical or similar functions across various aspects.

Hereinafter, exemplary embodiments of the present disclosure will be described in more detail with reference to the accompanying drawings.

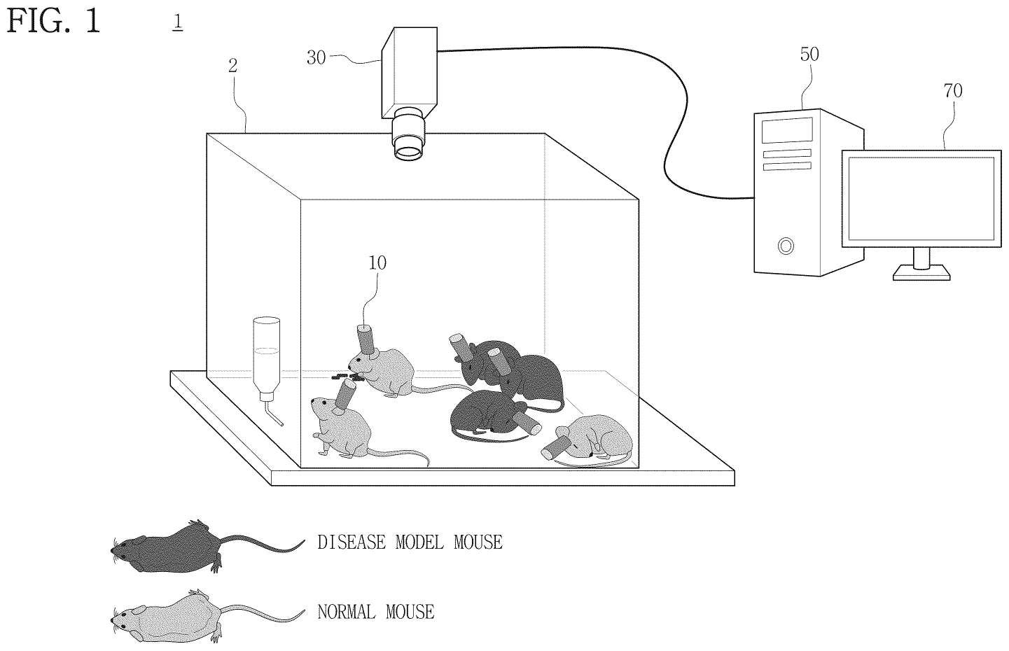

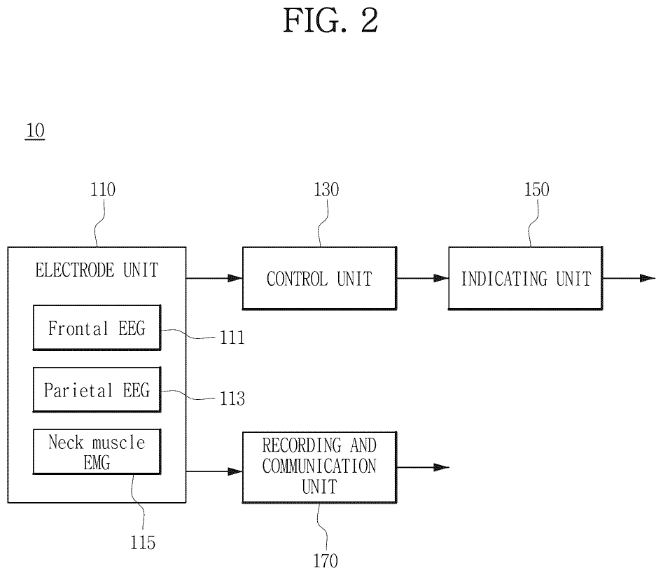

FIG. 1 is a schematic diagram of a system for monitoring a sleep state of a community according to the present disclosure. FIG. 2 is a block diagram of a device for monitoring a sleep state of a community according to one embodiment of the present disclosure.

A device 10 for monitoring a sleep state of a community (hereinafter referred to as “device”) in the community according to the present disclosure may be included in a system 1 for monitoring a sleep state of a community (hereinafter referred to as “system”) installed in a predefined test space 2 in which the community is present.

Referring to FIG. 1, the system 1 according to the present disclosure includes the device 10 mounted on each of all measurement targets (e.g., a mouse) present in the test space 2 and an image collection unit 30 for collecting images of the all measurement targets present in the test space 2. Here, the image collection unit 30 may be a photographing unit such as a camera or video camera.

In addition, the system 1 according to the present disclosure may further include a computer 50 for analyzing sleep patterns of the community using biosignals collected by the device 10 and image data collected by the image collection unit 30, and a display device 70 for displaying the analyzed data.

In FIG. 1, a mouse is illustrated as an example of the measurement target, and a disease model mouse and a normal mouse form one community, but the measurement target is not limited to the mouse, and as needed, the same type of test objects with various characteristics may be selected.

Referring to FIG. 2, the device 10 includes an electrode unit 110, a control unit 130, an indicating unit 150, and a recording and communication unit 170.

The device 10 according to the present disclosure may be mounted on a living test target to enable an unconstrained test, may monitor biosignals, and classify sleep and wake states of the measurement target according to critical values based on this.

The sleep and wake states causes changes in almost all physiological signals (body temperature, heart rate, blood pressure, respiration, blood sugar, electrocardiogram, EMG, EEG, and the like) including traditional physiological indicators, and these changes are expected to affect sleep regulation in a natural environment.

Since the electroencephalogram and electromyography are essential for determining the sleep state, in the present disclosure, it is essential to measure the two biosignals, and if necessary, an extra channel capable of measuring other biosignals may be further included.

In addition, since a sleep state of an entity (e.g., a mouse) lasts from several minutes to several tens of minutes and a time from waking up to falling asleep is required, measurement may be performed for a long time (e.g., 6 to 12 hours).

Software (application) for monitoring the sleep state of the community may be executed by being installed in the device 10 of the present disclosure, and the components of the electrode unit 110, the control unit 130, the indicating unit 150, and the recording and communication unit 170 may be controlled by the software for monitoring the sleep state of the community, which is executed in the device 10.

The device 10 may be a separate terminal or partial module of the terminal. In addition, the components of the electrode unit 110, the control unit 130, the indicating unit 150, and the recording and communication unit 170 may be formed as an integrated module or composed of one or more modules. However, conversely, each component may be configured as a separate module.

The electrode unit 110 measures biosignals including an EEG and EMG of each measurement target forming the community. In the present disclosure, sensors for measuring potential differences measured from one or more brain regions in a state in which entities freely move.

For example, the electrode unit 110 may include a probe 111 for measuring a frontal electroencephalogram (EEG) value, a probe 113 for measuring a parietal EEG value, a probe 115 for measuring a neck muscle electromyography (EMG) value, and the like.

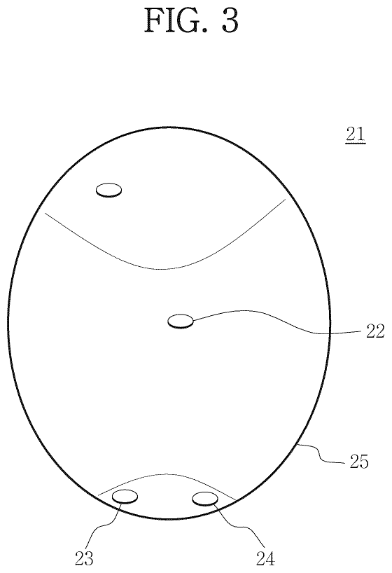

To this end, referring to FIG. 3, electrodes for installing electrodes on the brainpan of each test object may be positioned. FIG. 3 exemplarily illustrates an electrode 21 positioned on a frontal part, an electrode 22 positioned on a brain region near a parietal part or hippocampal part, a reference electrode 23 positioned at an edge of the brainpan, a ground electrode 24, and a tungsten thread 25 positioned on a neck muscle part.

The frontal part is a region in which a sleep slow wave, which is a specific waveform of NREM sleep, is best measured, and for example, since a mouse has the hippocampal part positioned under the parietal part, a specific theta wave of REM sleep is best measured from the parietal part.

The neck muscle is to measure muscle tension that varies depending on sleep and wake states. In one embodiment, the following equations can be established for the EEG value measured from the frontal part, the EEG value measured from the parietal part, and the neck muscle EMG value measured from the neck muscle based on each reference value.

Frontal EEG=Frontal screw−Reference

Parietal EEG=Parietal screw−Reference

Neck muscle EMG =Tungsten−Reference

The positions of the electrodes in FIG. 3 are only one example, and electrode units may be installed to be added and changed on sites necessary to measure the biosignals of the measurement target.

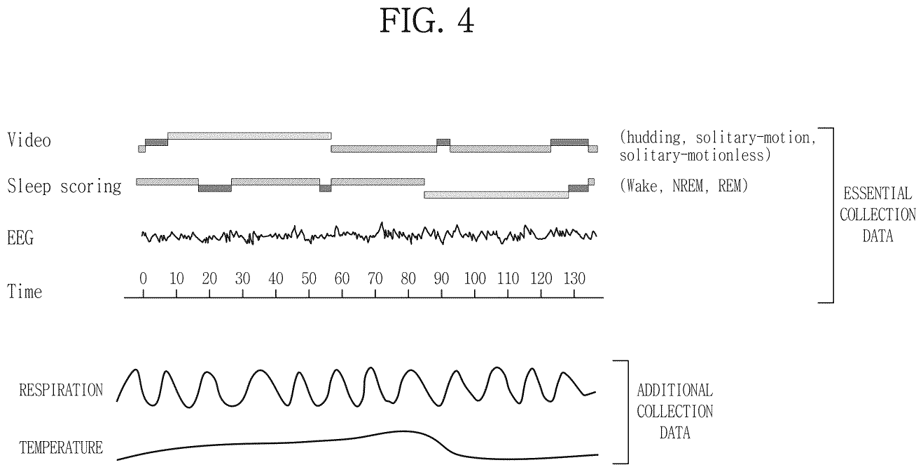

In addition, as illustrated in FIG. 4, the electrode unit 110 may further collect necessary biosignals such as respiration, body temperature, heart rate, blood pressure, respiration, blood sugar, and electrocardiogram of each measurement target as needed.

Conventional offline analysis has difficulty in detecting a change in sleep state for a short time, mainly at 4 to 10 second intervals. However, in the present disclosure, a change in sleep state for a short time of 1 to 2 seconds may also be detected by a change in EMG.

In addition, other biosignals for post-analysis may be collected simultaneously and may also be used for the polysomnographic evaluation for sleeping entities of a community.

The control unit 130 compares the measured EEG and EMG with critical values and classifies the sleep and wake states of the measurement target.

In order to distinguish the sleep and wake states and sleep stages in animals including humans, it is common for experts to manually analyze the sleep and wake states and sleep stages using EEG and EMG signals as essential indicators, or mechanically analyze the sleep and wake states and sleep stages based on sleep-specific frequencies shown in EEG.

Since the sleep-specific EEG show frequency characteristics of about 0.5 to 4 Hz, in order to analyze the sleep-specific EEG in real time, there is a need for a processing operation of storing signals for 2 seconds or more, analyzing frequencies, then comparing result values, and determining the sleep and wake states. Unlike the EEG, since the EMG is quickly changed by movement, signals of about 0.2 seconds may be stored and analyzed to increase the accuracy of real-time analysis.

In order to determine the sleep and wake states in real time based on a result of analyzing the frequencies, the critical value-based classification of the sleep EEG, which is commonly used in post-analysis, may be adopted.

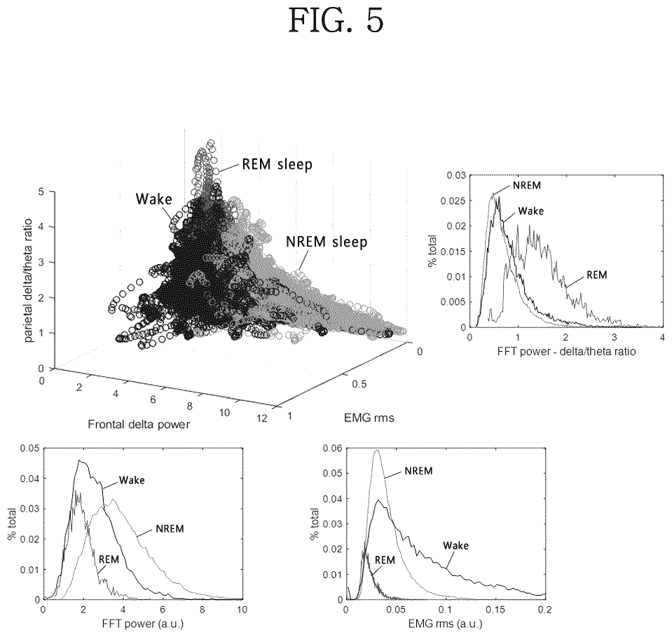

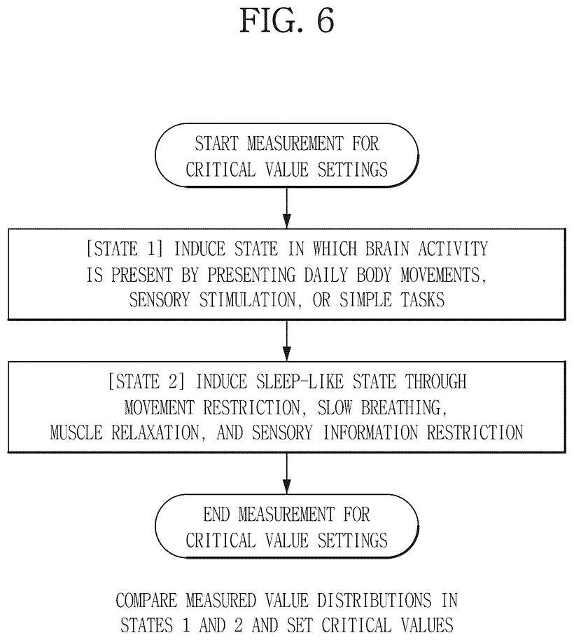

FIG. 5 is a graph for describing that a control unit of FIG. 2 sets a critical value. FIG. 6 is a view for describing a process of setting the critical value by the control unit of FIG. 2.

Referring to FIG. 5, it can be seen that the frequency and EMG analysis result values show differences specifically in wake/NREM sleep/REM sleep states. In addition, as illustrated in FIG. 6, an algorithm that immediately determines the sleep and wake states through a typical method of classifying the sleep and wake states by setting a critical value for each state can be applied.

In this case, the difficulty in which the size of the critical value is changed by a difference between individual entities and surgical effects can be resolved by pre-measuring the individual entities for a short time (e.g., 5 minutes or 1 hour) in a controlled state a few days before the test starts and setting the critical value for each entity using distribution values of the pre-measured values.

In particular, group research is possible when the present disclosure is used. In other words, an entity's sociality, a gathering behavior including hurdling, and sleep may be observed at the same time, and an environment may be controlled by allowing all entities to experience one environment. Therefore, it is possible to review the significance of the drug through group comparison through a single test and derive the results through only images without post-analysis that requires long hours of labor.

To this end, the control unit 130 may include a microprocessor capable of performing simple calculations (FFT, root mean square (RMS), ratio, and the like) and classification according to conditions on signals within 10 seconds in real time. For example, the EEG value may be derived by the FFT calculation, the EMG value may be derived by the RMS calculation, and each of the EEG and EMG values may be compared with each critical value.

In one embodiment, the control unit 130 may use programmed and pre-stored critical values (manual mode). In other words, a critical value of a classification condition for each entity is programmed into a device, and sleep is signed by changing colors of a light emitting diode (LED) according to conditions.

For convenience, the critical value may be set by measuring and then comparing signals in states with differences in the presence or absence of movement and brain activity for a short time (several minutes). In the case of animals, short-term inhalation anesthesia may be used for convenience.

In another embodiment, the control unit 130 may use critical values by setting the critical values based on biosignals measured for a predetermined time (auto mode). In this case, the control unit 130 may update each critical value at a predetermined period and use a fixed critical value when the critical value is not out of a preset range.

Referring to FIGS. 5 and 6, critical values for classifying wake/NREM/REM states using unsupervised classification with the signals stored in the device 10 may be automatically set.

For example, data measured from the moment the program is turned on may be divided into predetermined time units and stored as values (an EMG amplitude and an FFT value of an EEG) usually used to determine the sleep and wake states, and then when the data is divided into three types of clusters using a density-based clustering method, a boundary value of the clusters may be set to the critical value.

As illustrated in FIG. 6, in order to set the critical value, state 1 in which brain activity is present is induced by presenting daily body movements, sensory stimulation, or simple tasks, and in contrast, sleep-like state 2 is induced through movement restriction, slow breathing, muscle relaxation, and sensory information restriction. Therefore, the critical values may be set by comparing the distribution of measured values in state 1 and state 2.

For example, classification may be set to be performed using data for at least 3 hours. In addition, when the critical value changes to a predetermined level or less, the update may be stopped to fix the critical value, and the fixed critical value may be used for a test. The fixed critical value should be a value in a pre-specified biosignal range, and when the sleep and wake states are divided by the critical values, the critical value setting may be designated as a completed state when a naturally possible ratio (e.g., 40% wake, 10% REM sleep, or 50% NREM sleep) is met.

In addition, while the critical values are being updated, the update of the critical value may be indicated by using the indicating unit 150. For example, in order to indicate that the critical value is being updated, the critical values which is being updated may be indicated by adjusting the LED light to be brighter during the update or adding a white LED, indicate that the update is being updated.

The control unit 130 may determine that when the measured EMG value of the measurement target is larger than a first critical value, the measurement target is in a wake state and determine that when the measured EMG value of the measurement target is smaller than a second critical value and the EEG value of the parietal part is larger than a third critical value, the measurement target is in a REM sleep state. In this case, the first critical value may have a larger value than the second critical value.

It may be determined that when the measured EMG value of the measurement target is smaller than the second critical value and the frontal EEG value is smaller than a fourth critical value, the measurement target is in a NREM sleep state, and it may be determined that when the measured EMG value of the measurement target is smaller than the first critical value and the frontal EEG value is larger than the fourth critical value, the measurement target is in the NREM sleep state.

In addition, the control unit 130 may determine that when the measured EMG value of the measurement target is larger than or equal to the first critical value and the frontal EEG value is smaller than or equal to the fourth critical value, the measurement target is in a wake state or unknown state.

The indicating unit 150 emits different colors according to the sleep and wake states of the measurement target.

The behavior and position of the measurement entity may be easily observed through a video, and many entity behavior test paradigms that analyze the video and report the behavior are present. In order to observe the sleep and wake states determined by the EEG and the video behavior together, there is the inconvenience of having to synchronize a behavior video with EEG measurement data and handle two different data.

In order to immediately observe changes in sleep and wake states due to environmental changes, stimulation, and the like and measure the unlimited number of entities in a wide space, there is a need for a method of observing the sleep and wake states as well as video data.

To this end, in the present disclosure, the sleep and wake states may be visualized to be observed through the video unit (see FIG. 1), and various sleep and wake states may be indicated by using various colors of LED light on a surface of a detector of the unconstrained EEG measurement device.

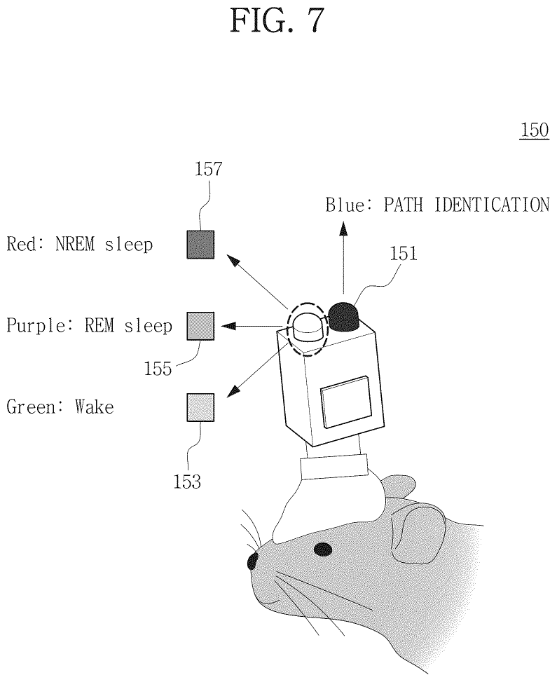

Referring to FIG. 7, the indicating unit 150 may include a first light emitting part 151 for indicating a position of a measurement target, a second light emitting part 153 for indicating a wake state, a third light emitting part 155 for indicating a REM sleep state, and a fourth light emitting part 157 for indicating a NREM sleep state.

Each of the first light emitting part 151, the second light emitting part 153, the third light emitting part 155, and the fourth light emitting part 157 may emit light in different colors and include, for example, an LED.

In FIG. 7, the first light emitting part 151 for indicating the position of the measurement target emits blue light, and the second light emitting part 153, the third light emitting part 155, and the fourth light emitting part 157 indicate the wake state, the REM sleep state, and the NREM sleep state, respectively, and emit light in green, purple, and red, respectively. However, this is only one example and may be changed as needed.

In the present disclosure, the position (sleeping position, whether the measurement entities are gathered, and the like) of the measurement entity may be identified by analyzing the images collected by the image collection unit 30, and the community sleep patterns may be analyzed by detecting a change in LED color of the indicating unit 150.

The recording and communication unit 170 stores or transmits the measured EEG and EMG to an external device.

Since a sleep state may be determined through an EEG during sleeping and the EEG during sleeping also include waveforms related to cognitive/physiological factors, the EEG should be stored to enable post-analysis for specific EEG analysis of an individual entity.

The recording and communication unit 170 may transmit EEG measured values to the computer 50 using Bluetooth communication or store the EEG measured values in an internal memory. To this end, the recording and communication unit 170 may include an amplifier, an embedded memory, and the like or store data in the computer 50 via Bluetooth communication.

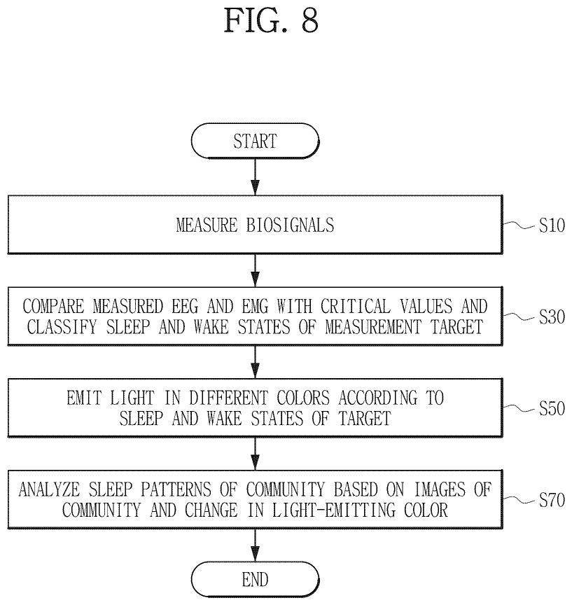

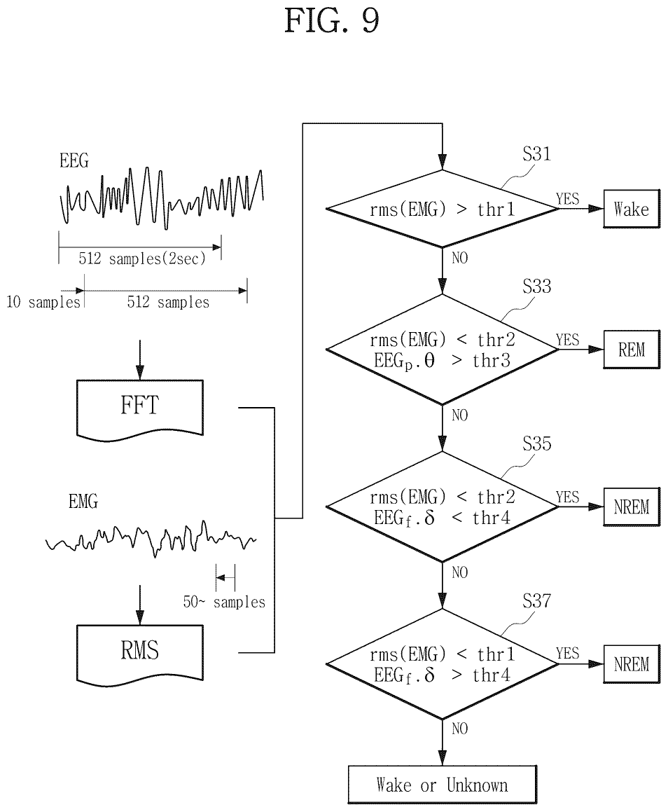

FIG. 8 is a flowchart of a method of monitoring a sleep state of a community according to one embodiment of the present disclosure. FIG. 9 is a detailed flowchart of a process of classifying sleep and wake states of a measurement target using critical values in FIG. 8.

The method of monitoring mutual activity between brains and objects in a community according to the present embodiment may be performed in a component that is substantially the same as the system 1 of FIG. 1 and the device 10 of FIG. 2.

In other words, in the present disclosure, the method may be performed by the device 10 of FIG. 2 mounted on each measurement target forming the community in the system 1 for monitoring the sleep state of the community installed in the predefined test space 2.

Therefore, a component that is the same as the system 1 of FIG. 1 and the device 10 of FIG. 2 are denoted by the same reference numeral, and repeated description thereof will be omitted.

The present disclosure relates to a method of allowing the device 1 mounted on the living measurement target to conduct the unconstrained test, monitoring the biosignals, and classifying the sleep and wake states of the measurement target according to the critical values based on this.

Referring to FIG. 8, the method of monitoring the mutual activity between the brains and objects in the community according to the present embodiment includes measuring biosignals including an EEG and an EMG of the measurement target using the device for monitoring the sleep state of the community mounted on each measurement target forming the community (operation S10).

In the measuring of the biosignals, if necessary, respiration, body temperature, heart rate, blood pressure, respiration, blood sugar, electrocardiogram, and the like of each measurement target may be additionally measured and used for post-analysis.

To this end, sensors for measuring potential differences measured from one or more brain regions in a state in which the measurement target (e.g., a mouse) freely moves may be included.

In the present disclosure, a change in sleep state for a short time of 1 to 2 seconds may be detected through a change in EMG, and other biosignals for post-analysis may be simultaneously collected and used for polysomnographic evaluation of community sleep entities.

The measured EEG and EMG are compared with the critical values, and the sleep and wake states of the measurement target are classified (operation S30). For example, the EEG value may be derived by the FFT calculation, the EMG value may be derived by the RMS calculation, and each of the EEG and EMG values may be compared with each critical value.

Referring to FIG. 9, in the classifying of the sleep and wake states of the measurement target, when the measured EMG value of the measurement target is larger than the first critical value, it is determined that the measurement target is in a wake state (operation S31).

When the measured EMG value of the measurement target is smaller than the second critical value and the parietal EEG value is larger than the third critical value, it is determined that the measurement target is in a REM sleep state (operation S33). Here, the first critical value may have a larger value than the second critical value.

When the measured EMG value of the measurement target is smaller than the second critical value and the frontal EEG value is smaller than the fourth critical value, it is determined that the measurement target is in a NREM sleep state (operation S35).

When the measured EMG value of the measurement target is smaller than the first critical value and the frontal EEG value is larger than the fourth critical value, it is determined that the measurement target is in the NREM sleep state (operation S37).

When the measured EMG value of the measurement target is larger than or equal to the first critical value and the frontal EEG value is smaller than or equal to the fourth critical value, it is determined that the measurement target is in a wake state or unknown state.

In one embodiment, in the classifying of the sleep and wake states of the measurement target, the programmed and pre-stored critical values may be used (manual mode). In other words, a critical value of a classification condition for each entity is programmed into a device, and sleep is indicated by changing colors of an LED according to conditions.

In another embodiment, in the classifying of the sleep and wake states of the measurement target, the critical values may be used by being set based on the biosignals measured for the predetermined time (auto mode). In this case, the control unit 130 may update each critical value at a predetermined period and use a fixed critical value when the critical value is not out of the preset range.

Specifically, the critical values may be set based on the biosignals measured for the predetermined time, and each critical value may be updated at the predetermined period. Then, when each critical value is not out of the preset range, the critical value may be used by being fixed.

For example, for convenience, critical value settings for each entity may be performed through measurement for a short time (within about 10 minutes) using inhalation anesthesia. For example, the update of the critical value may be set in 30-minute units, and when the critical value changes to a predetermined level or less, the update may be stopped to fix the critical value, and the fixed critical value may be used in the test.

Light is emitted in different colors according to the sleep and wake states of the measurement target (operation S50).

For example, light may be emitted in a first light-emitting color for indicating the position of the measurement target. In addition, light may be emitted in a second light-emitting color for indicating the wake state, and light may be emitted in a third light-emitting color for indicating the REM sleep state. In addition, light may be emitted in a fourth light-emitting color for indicating the NREM state.

For example, light may be emitted in blue to indicate the position of the measurement target, and light may be emitted in green, purple, and red to indicate a wake state, REM sleep state, and NREM sleep state, respectively. However, this is only one example and may be changed as needed.

Furthermore, light may be emitted in a fifth light-emitting color during the update of the critical value. For example, in order to indicate that the critical value is being updated, the critical values which is being updated may be indicated by adjusting the LED light to be brighter during the update or adding a white LED, indicate that the update is being updated.

The sleep patterns of the community are analyzed based on the images of the community and the change in light-emitting color (operation S70).

Since the sleep state may be determined through the EEG during sleeping and the EEG during sleeping also include the waveforms related to the cognitive/physiological factors, the EEG should be stored to enable the post-analysis for specific EEG analysis of the individual entity. Therefore, the measured EEG and EMG may be stored or transmitted to an external device.

In the present disclosure, the position (sleeping position, whether the measurement entities are gathered, and the like) of the measurement entity may be identified by analyzing the images photographing the community, and the community sleep patterns may be analyzed by detecting a change in light-emitting color (LED color).

The method of monitoring the sleep state of the community can be implemented as an application or implemented in the form of program commands that may be executed through various computer components and recorded on a computer-readable recording medium. The computer-readable recording medium may include program commands, data files, data structures, and the like alone or in combination.

The program commands recorded on the computer-readable recording medium may be specially designed and configured for the present disclosure or may be known and available to those skilled in the field of computer software.

Examples of the computer-readable recording medium include magnetic media such as a hard disk, a floppy disk, and a magnetic tape; optical recording media such as a CD-ROM and a DVD; magneto-optical media such as a floptical disk; and hardware devices specifically configured to store and perform program commands, such as a read-only memory (ROM), a random-access memory (RAM), and a flash memory.

Examples of the program commands include not only machine language code such as that produced by a compiler, but also high-level language code that may be executed by a computer using an interpreter or the like. The hardware device may be configured to operate as one or more software modules to perform processing according to the present disclosure and vice versa.

Although the present disclosure has been described above with reference to the embodiments, those skilled in the art will understand that the present invention may be modified and changed variously without departing from the spirit and scope of the present disclosure as described in the appended claims.

Global neuromonitoring market size is expected to grow from $1.32 billion in 2020 to $1.67 billion by 2023, growing at a CAGR of 4.9%. In particular, global electroencephalography (EEG) market size is expected to grow from $605 million in 2020 to $960 million by 2025, growing at a CAGR of 9.0%. The present disclosure is expected to be used in the neuromonitoring and EEG markets.

What is claimed is:

1. A device for monitoring a sleep state of a community mounted on each measurement target forming the community, comprising:
an electrode unit configured to measure biosignals including an electroencephalogram (EEG) and electromyography (EMG) of each measurement target forming the community;
a control unit configured to compare the measured EEG and EMG with critical values and classify sleep and wake states of the measurement target;
an indicating unit configured to emit light in different colors according to the sleep and wake states of the measurement target; and
a recording and communication unit configured to store or transmit the measured EEG and EMG to an external device.

2. The device of claim 1, wherein the control unit is configured to:
determine that when a value of the measured EMG of the measurement target is larger than a first critical value, the measurement target is in a wake state;
determine that when the measured EMG value of the measurement target is smaller than a second critical value and a parietal EEG value is larger than a third critical value, the measurement target is in a REM sleep state;
determine that when the measured EMG value of the measurement target is smaller than the second critical value and a frontal EEG value is smaller than a fourth critical value, the measurement target is in a NREM sleep state; and
determine that when the measured EMG value of the measurement target is smaller than the first critical value and the frontal EEG value is larger than the fourth critical value, the measurement target is in the NREM sleep state.

3. The device of claim 2, wherein the control unit determines that when the measured EMG value of the measurement target is larger than or equal to the first critical value and the frontal EEG value is smaller than or equal to the fourth critical value, the measurement target is in the wake state or unknown state.

4. The device of claim 2, wherein the first critical value has a larger value than the second critical value.

5. The device of claim 1, wherein the control unit uses programmed and pre-stored critical values.

6. The device of claim 1, wherein the control unit uses the critical values by setting the critical values based on biosignals measured for a predetermined time.

7. The device of claim 6, wherein the control unit updates each critical value at a predetermined period and uses a fixed critical value when the critical value is not out of a preset range.

8. The device of claim 7, wherein the update of the critical value is indicated by using an indicating unit while the critical values are being updated.

9. The device of claim 1, wherein the indicating unit includes:
a first light-emitting unit configured to indicate a position of the measurement target;
a second light-emitting unit configured to indicate the wake state;
a third light-emitting unit configured to indicate the REM sleep state; and
a fourth light-emitting unit configured to indicate the NREM sleep state.

10. The device of claim 1, wherein the electrode unit further collects at least one biosignal among respiration, body temperature, heart rate, blood pressure, respiration, blood sugar, and electrocardiogram of each measurement target.

11. A method of monitoring a sleep state of a community, comprising:
measuring biosignals including an electroencephalogram (EEG) and electromyography (EMG) of a measurement target using a device for monitoring the sleep state of the community mounted on each measurement target forming the community;
comparing the measured EEG and EMG with critical values and classifying sleep and wake states of the measurement target;
emitting light in different colors according to the sleep and wake states of the measurement target; and
analyzing sleep patterns of the community based on images of the community and a change in light-emitting color.

12. The method of claim 11, wherein the classifying of the sleep and wake states of the measurement target includes:
determining that when a value of the measured EMG of the measurement target is larger than a first critical value, the measurement target is in a wake state;
determining that when the measured EMG value of the measurement target is smaller than a second critical value and a parietal EEG value is larger than a third critical value, the measurement target is in a REM sleep state;
determining that when the measured EMG value of the measurement target is smaller than the second critical value and a frontal EEG value is smaller than the fourth critical value, it is determined that the measurement target is in a NREM sleep state; and
determining that when the measured EMG value of the measurement target is smaller than the first critical value and the frontal EEG value is larger than the fourth critical value, the measurement target is in the NREM sleep state.

13. The method of claim 12, wherein the classifying of the sleep and wake states of the measurement target further includes determining that when the measured EMG value of the measurement target is larger than or equal to the first critical value and the frontal EEG value is smaller than or equal to the fourth critical value, the measurement target is in the wake state or unknown state.

14. The method of claim 11, wherein in the classifying of the sleep and wake states of the measurement target, programmed and pre-stored critical values are used.

15. The method of claim 11, wherein the classifying of the sleep and wake states of the measurement target includes:
setting critical values based on biosignals measured for a predetermined time;
updating each critical value at a predetermined period; and
fixing the critical value when each critical value is not out of a preset range.

16. The method of claim 11, wherein the emitting of the light in different colors according to the sleep and wake states of the measurement target includes:
emitting light in a first light-emitting color for indicating a position of the measurement target;
emitting light in a second light-emitting color for indicating the wake state;
emitting light in a third light-emitting color for indicating the REM sleep state; and
emitting light in a fourth light-emitting color for indicating the NREM sleep state.

17. The method of claim 16, wherein the emitting of the light in different colors according to the sleep and wake states of the measurement target further includes emitting light in a fifth light-emitting color during the update of the critical value.

18. The method of claim 11, further comprising storing or transmitting the measured EEG and EMG to an external device.

19. The method of claim 11, wherein in the measuring of the biosignals, at least one biosignal among respiration, body temperature, heart rate, blood pressure, respiration, blood sugar, and electrocardiogram of each measurement target is further collected.

20. A computer-readable non-transitory storage medium in which a computer program for performing the method of monitoring the sleep state of the community according to claim 11 is recorded.
